(19) United States
(12) Patent Application Publication
(10) Pub. No.: US 2011/0318756 A1
(43) Pub. Date: Dec. 29, 2011

(54) SMALL SPECIMEN STAINING AND DIAGNOSING OF CELLS

(75) Inventor: Peter Tsivis, Clearwater, FL (US)

(73) Assignee: C.V. DIAGNOSTICS LLC, CLEARWATER (US)

(21) Appl. No.: 13/143,234

(22) PCT Filed: Jan. 11, 2010
(86) PCT No.: PCT/US10/20667

§ 371 Date: Jul. 5, 2011

(30) Foreign Application Priority Data
Jan. 9, 2009 (US) 61/204763

(51) Int. Cl. G01N 33/577; G01N 33/566

(52) U.S. Cl. 435/7.21; 435/7.1

(57) ABSTRACT

An immunohistochemical staining of small specimen comprises using a plurality of antibodies and/or antigens to mark certain cells with particular colors of stains in order to distinguish target cells, such as carcinoma cells, in a stained small specimen. For example, antibodies CD44, cytokeratin 20 and p53 may be used for selectively staining a specimen of a urothelial mucosal biopsy on a single slide. Mouse monoclonal antibody CD44 is associated only with reactive urothelial cells, while rabbit monoclonal antibody p53 is associated only with carcinoma cells. Mouse monoclonal antibody cytokeratin 20 is associated with both “umbrella cells” and carcinoma cells, but antibody p53 is not associated with umbrella cells, which are the most superficial urothelial cells and are characterized morphologically from the other cells in a prepared specimen. Thus, diagnosis is facilitated by the staining of carcinoma cells in a contrasting color to normal urothelial cells and superficial urothelial cells.

### RELATED APPLICATION

This application is a National Stage of PCT/US2010/020667 filed on Jan. 11, 2010 which claims the benefit of the filing date of U.S. Provisional Application No. 61/204,763 filed Jan. 9, 2009, which is incorporated herein in its entirety, including the color micrographs of the application, which show the original staining of the micrographs schematically represented in FIGS. 1-4 of the present application.

### FIELD OF THE INVENTION

The field relates to the biopsy, sectioning, staining and cytology of cellular tissues.

### BACKGROUND

Cytokeratin 20 is a type 1 keratin that is expressed in some adenocarinomas, mucinous ovarian tumors, transitional cell carcinomas of the urinary tract, and Merkel cell carcinomas. Mouse monoclonal antibodies are known for cytokeratin 20, which is used to distinguish one type of cancer from a type of cancer for which cytokeratin 20 is not expressed.

CD44 mouse monoclonal antibodies, such as CD44 [P2A1], are available from GeneTex® and other sources, for example. Woodman et al., “Noninvasive diagnosis of bladder carcinoma by enzyme-linked immunosorbent assay detection of CD44 isoforms in exfoliated urothelia,” Clinical Cancer Research, 6:2381-2392 (2000) discloses that CD44 isoforms may be used to diagnose cancer using a Western Blot or ELISA assay. The reference teaches away from the use of microscopic cytology as an assay for detecting cancer using CD44 isoforms associated with bladder cancer.

The p53 tumor suppressor protein is involved in cellular response to DNA damage and other genomic aberrations in a wide variety of malignant tumors including breast, ovary, bladder, colon, lung, and melanoma. Many antigens are available for p53, such as p53 mAb (pAb122) mouse monoclonal antibody from Assay Designs. One of the problems identified by L. M. McShane, R. Aamodt, C. Cordon-Cardo, R. Cote, D. Faraggi, Y. Fradet, H. B. Grossman, A. Peng, S. E. Taube, F. M. Waldman, and t. N. C. I. B. T. M. Network, “Reproducibility of p53 Immunohistochemistry in Bladder Tumors,” Clin. Cancer Res. 2000 6:1854-1864 is the inconsistency between laboratory assessments, when p53 is indicated in an intermediate percentage of nuclei. Thus, use of p53, when at or near the threshold range for binary determination from nuclear staining, may not be consistent from one laboratory to the next. The authors attributed this discordance to variability in staining and variability in setting of the threshold. A variability in staining (brown) of nuclei is evidenced between the examples of FIG. 1 (dark brown staining) and FIG. 3 (tan or lighter brown staining), using the same protocol, antigen and staining agents. Variability is likely to be even greater if different protocols, antigens and staining agents are used for p53 assessment, as pointed out by the McShane reference. Thus, assessment of p53 as a cancer marker, while known and commonly used for a wide variety of uses, has significant shortcomings and limitations that complicate assessment and diminish any expectation of reliance on the assessment made using p53 for clinical diagnosis or in studies using data based on p53 assay.

In U.S. Pat. Publ. No. 2005/0186642, published Aug. 25, 2005, David Tacha discloses immunoassay reagents and methods of use for cytoloty using double and triple stain protocols, but Tacha fails to disclose any assay or method for urothelial cell carcinoma using specific antibodies associated with benign and cancerous cells.

### SUMMARY

An immunohistochemical staining of small specimen comprises using a plurality of antibodies and/or antigens to mark certain cells with particular colors of stains in order to distinguish target cells, such as carcinoma cells, in a stained small specimen. By staining normal cells one color, while staining abnormal or diseased cells another color, contrast between healthy and diseased cells becomes readily apparent. In one example, the staining is conducted on a sample on a single slide by mixing a cocktail of markers, such as antibodies that associate with some cells but not others, and staining the specimen after exposure to the antibodies, which causes a color change for only certain targeted cells and not others.

For example, antibodies CD44, cytokeratin 20 and p53 may be used for selectively staining a specimen of a urothelial mucosal biopsy on a single slide. Mouse monoclonal antibody CD44 is associated only with reactive urothelial cells, while rabbit monoclonal antibody p53 is associated only with carcinoma cells. Mouse monoclonal antibody cytokeratin 20 is associated with both “umbrella cells” and carcinoma cells, but antibody p53 is not associated with umbrella cells, which are the most superficial urothelial cells and are characterized morphologically from the other cells in a specimen prepared using this triple immunostain technique. Thus, diagnosis is facilitated by the triple immuno-staining of carcinoma cells in a color contrasting with the color(s) of normal urothelial cells and superficial urothelial cells, by targeting antibodies/antigens unique to the carcinoma cells.

### BRIEF DESCRIPTION OF THE DRAWINGS

The application file contains at least one drawing executed in color. Copies of this patent application publication with color drawing(s) will be provided by the Office upon request and payment of the necessary fee.

FIG. 1 illustrates a photomicrograph of a stained specimen.

FIG. 2 illustrates a detailed view of another photomicrograph of a stained specimen at a higher magnification than FIG. 1.

FIG. 3 illustrates yet another photomicrograph of a stained specimen at a higher magnification than FIG. 1.

FIG. 4 illustrates still another photomicrograph of a stained specimen at a higher magnification than FIG. 1.

FIG. 5 illustrates the schematic structure of CD44 with arrows indicating antibody binding domains.

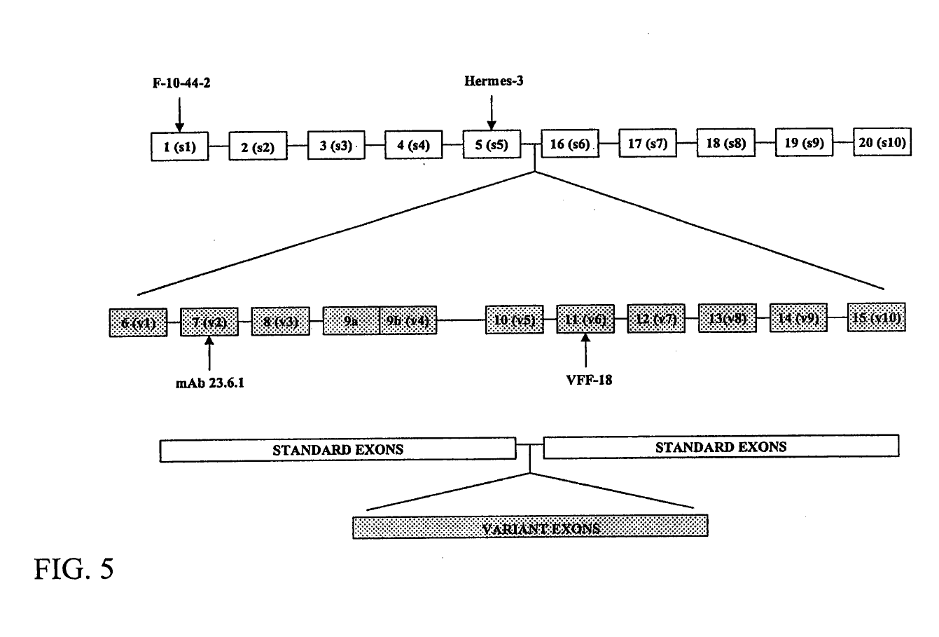

### DETAILED DESCRIPTION

Examples of staining of small specimens for the detection of diseased cells are described; however, the examples described and the photomicrographs presented are merely examples of the present invention. The claims that eventually issue should be interpreted in light of the specification, but the claims should not be limited by the description and drawings of the examples presented.

In one example, the following steps are performed to triple immuno-stain a specimen of urothelial cells on a standard formalin fixed paraffin embedded tissue section of a urothelial mucosal biopsy. First, the specimen is deparaffinized, and the tissue section is rehydrated. Then, the unstained tissue section, or a plurality of sections, may be heated, such as in a digitally programmable pressure cooker of the type sold by Biocare, with a retrieval solution at 125 degrees Celsius for 30 minutes, for example. A TBS wash buffer may be used to rinse the slide, following the incubation period, and a CD44 mouse monoclonal antibody/p53 rabbit monoclonal antibody cocktail may be deposited onto the specimen, which may be allowed to remain on the specimen at room temperature (20-25 degrees Celsius) for 30 minutes.

Again, the specimen may be rinsed with a TBS wash buffer before depositing a double stain polymer detection kit #1, mouse alkaline phosphatase/rabbit horseradish peroxidase (Biocare) onto the specimen, which may be allowed to remain on the specimen at room temperature for another 30 minutes, for example. Then, after rinsing with a TBS wash buffer again, a diaminobenzidine (DAB) chromogen, such as Betazoid™ DAB—Biocare, may be deposite onto the specimen, which may remain at room tissue for 5 minutes, for example.

Again, after rinsing with TBS wash buffer, a blue chromogen, such as Ferengi Blue™ chromogen—Biocare, may be deposited onto the specimen, which may remain at room temperature for 10 minutes. The slide may be rinsed with deionized water, and a danaturing solution may be deposited onto the specimen at a 1:4 dilution, which may remain on the specimen at room temperature for 3 minutes before rinsing the specimen with a TBS wash buffer.

A cytokeratin 20 mouse monoclonal antibody may be deposited onto the specimen and may be allowed to remain in contact with the specimen at room temperature for 30 minutes prior to rinsing the specimen, again, with a TBS wash buffer. Subsequently, a polymer—alkaline phosphatase detection conjugate, e.g. of Biocare, may be deposited onto the specimen, which may be allowed to remain at room temperature for 30 minutes before rinsing with a TBS wash buffer. Staining proceeds with the depositing of a red chromogen, such as the Vulcan Fast Red™ chromogen of Biocare, onto the specimen and leaving it in contact with the specimen at room temperature for 5 minutes before rinsing the specimen with TBS wash buffer. Finally, the specimen may be lightly counterstained, such as with hematoxylin, by contacting the specimen with the counterstain at room temperature for 1 minute. The stained specimen may have a coverslip applied to the slide, such as with a SlideBrite™ coverslip.

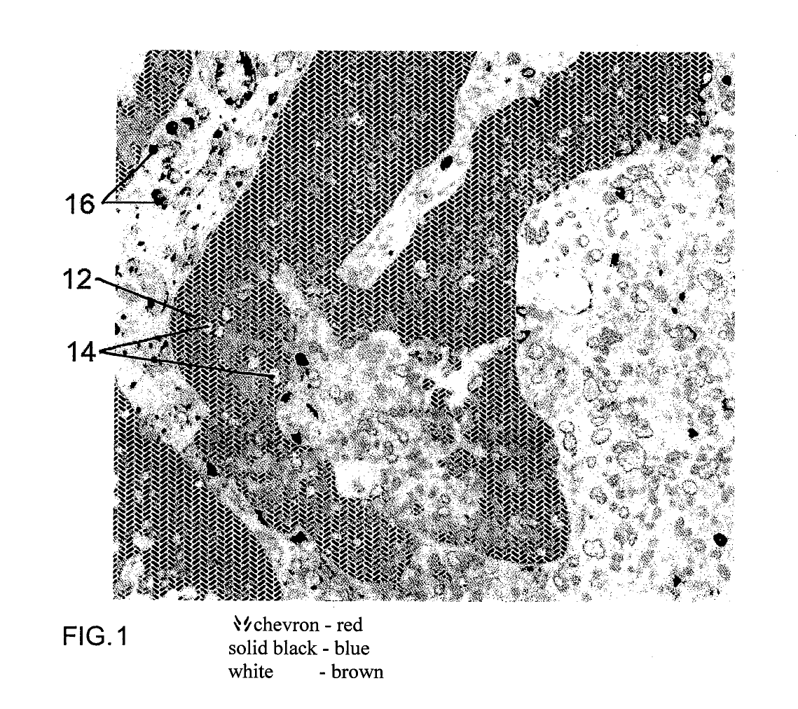

An example of a specimen prepared according to this example of the method of immuno-staining is shown in the photomicrograph of FIG. 1. In FIG. 1, taken at a microscopic magnification of 10×, a population of urothelial cancer cells demonstrate red cytoplasmic staining 12 for cytokeratin 20 and brown nuclear staining 14 for p53. These cells are readily identifiable from surrounding cells. Focal punctate blue staining 16 for CD44 is noted in a few residual benign cells at the base of the urothelium.

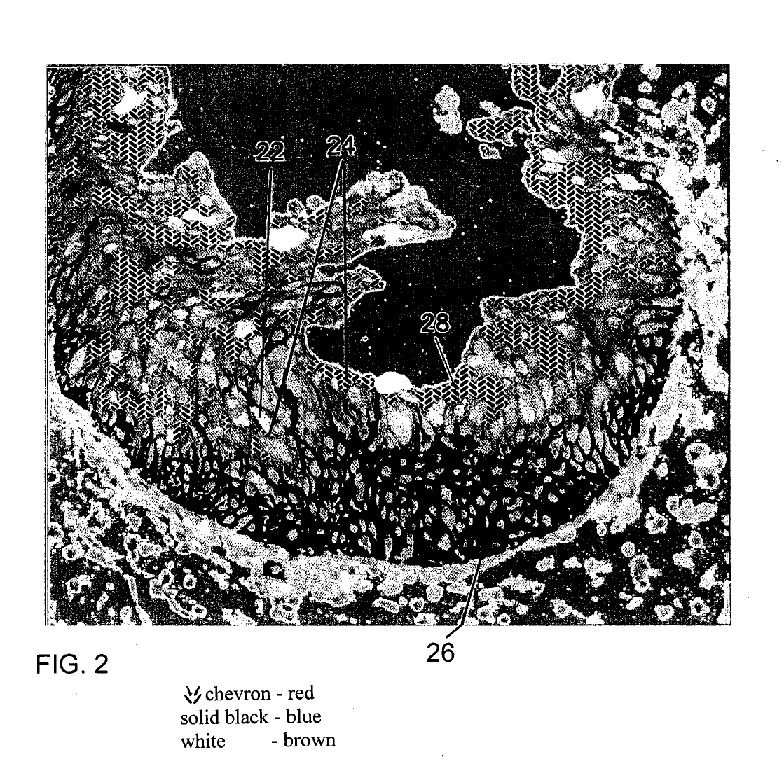

Another example of a specimen prepared according to this example of the method of immuno-staining is shown in the photomicrograph of FIG. 2, which is a detailed view at a higher magnification. The detailed view of FIG. 2 illustrates a photomicroph showing a high contrast between the urothelium cancer cells (red cytoplasm 22-brown nuclei 24) and a large population of reactive urothelium cytoplasm stained blue 26. While a superficial layer of urothelial cells, the so-called umbrella cells 28, stain positive for cytokeratin 20, these cells do not stain with p53 and are morphologically readily recognizable as benign by a pathologist.

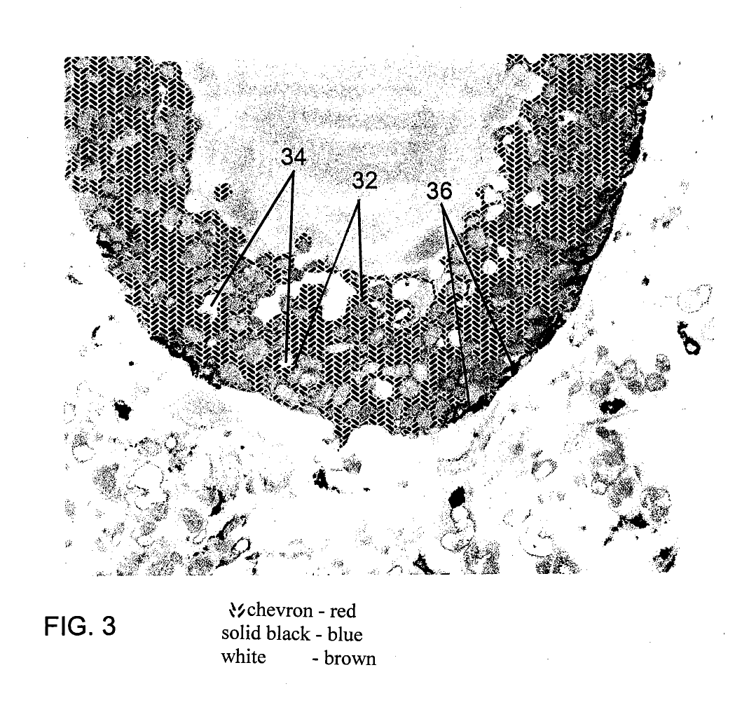

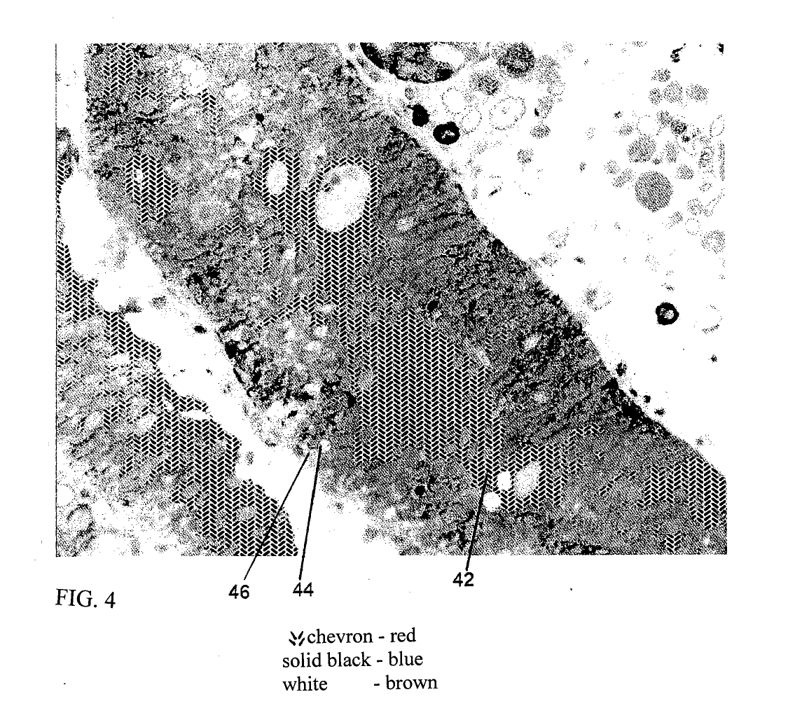

In contrast, a known process for staining of urothelial cells results in photomicrographs having a uniform brown staining of various structures associated with mutagenic cells. By comparing various sections, before and after staining of certain chromogens, cancerous cells may be distinguished from benign cells and one cancer type may be distinguished from another, for example. In comparison, the micrographs of FIGS. 1 and 2 clearly distinguish normal from carcinoma cells in the biopsy, greatly reducing the likelihood of a missed diagnosis. Surprisingly, the results of assays performed using examples of the methods are unexpectedly good at resolving discrepancies when an intermediate range of cells demonstrate mutagenisis of one of the target markers, such as illustrated in the photomicrographs of FIGS. 2-4. For example, FIG. 3 illustrates another detailed view of a photomicraph at a higher magnification than in FIG. 1. In FIG. 3 the urothelial cancer cells of the carcinoma in situ are readily identified as penetrating the full thickness of the urothelium, even though staining of the nuclei is fainter than in FIG. 1. FIG. 4 illustrates a specimen having a few carcinoma cells; however, in FIG. 4 there are cells identified as benign by demonstrating a blue chromogen in the cytoplasm, while being identified as cancerous by demonstrating a brown chromogen in the nuclei. This discrepancy is readily identified by a pathologist. A more careful consideration of the reasons for such a result may be preferred. For example, such a result may be caused by tangential sectioning or protocol errors, which might require resectioning and staining of a new specimen or examination of other sections taken from a biopsy. The use of the three contrasting chromogens, each with a different color and targeting a different protein marker for both benign and malignant cellular structures, provides for a much easier and less subjective diagnosis by a pathologist and/or an automated cytological analysis.

In one example, the percentage of blue, red and brown are automatically determined using an image analysis system. A threshold value or values may be defined to determine when an automated assay suggests a diagnosis. In one example, an automated system may flag an image as unresolved, requiring a pathologist to review the image before providing a recommendation. For example, an image analyzer and optical magnification system may be coupled with a charge coupled device and a flame grabber managed by a computer to determine the relative or absolute colors within a field of view or a series of fields of view, at a single magnification or a series of magnifications, in order to determine, automatically, if a prepared specimen indicates cytological pathology indicating disease, no disease or an indeterminate status.

Combinations and variations of a test kit, use of the components of an assay and the method of small specimen staining and diagnosis provided in the examples of the detailed description will be readily apparent to a person having ordinary skill in the art, based on this disclosure. Combinations and variations to the examples are included within the scope of the invention, and any claims that eventually issue should not be limited to the specific examples provided.

What is claimed is:

1. An immunohistochemical staining kit for staining a specimen, the kit comprising:
a plurality of selectively binding agents made of antibodies, antigens or a combination of antibodies and antigens, the selectively binding agents being selected to bind to certain features in cells of the specimen such that a first one of the plurality of selectively binding agents targets a certain feature of abnormal cells, preferentially, and a second one of the plurality of selectively binding agents targets a certain feature of one of the normal healthy cells, preferentially; and
a plurality of stains selected to impart a first color, preferentially, to the certain feature of abnormal cells targeted by the first one of the plurality of selectively binding agents, and to impart a second color, contrasting with the first color, preferentially, to the certain features of one of the normal, healthy cells targeted by the second one of the plurality of selectively binding agents, such that contrast between the first color and the second color distinguishes the abnormal cells from the normal, healthy cells.

2. The kit of claim 1, further comprising a third one of the plurality of selectively binding agents selected to target another feature of another one of the normal, healthy cells; and
the plurality of stains are selected to impart a third color, contrasting with both the first color and second color, such that the another one of the normal, healthy cells is distinguishable from the certain feature of abnormal cells and the certain feature of one of the normal, healthy cells.

3. The kit of claim 2, wherein the one of the normal, healthy cells includes urothelial cells and the another of the normal, healthy cells includes umbrella cells, and the third one of the plurality of selectively binding agents is selected to target another feature of the umbrella cells; and the third color imparted by the plurality of stains stains a portion of the umbrella cells, such that the umbrella cells are distinguishable by color contrast from the one of the normal, healthy cells and the abnormal cells.

4. The kit of claim 3, wherein the third one of the plurality of selectively binding agents is monoclonal antibody cytokeratin 20.

5. The kit according to claims 1-4, wherein the first one of the plurality of selectively binding agents is monoclonal antibody p53.

6. The kit according to claims 1-5, wherein the second one of the plurality of selectively binding agents is monoclonal antibody CD44.

7. The kit according to claims 1-6, wherein the plurality of stains includes a diaminobenzidine chromogen, a blue chromogen and a red chromogen.

8. The kit according to claims 1-7, wherein the plurality of stains includes hematoxylin.

9. An automated device for staining a specimen, the device comprising the kit of claim 1.

10. A process for staining specimen using the kit of claim 2, the process comprising:
depositing a cocktail of the first one of the plurality of selectively binding agents and the second one of the plurality of selectively binding agents onto the specimen;
rinsing the specimen;
selecting a first chromogen and depositing it on a surface of the specimen rinsing the surface of the specimen;
selecting a second chromogen and depositing it on the surface of the specimen;
rinsing the surface;
selecting and depositing the third one of the plurality of selectively binding agents on the specimen;
rinsing the specimen;
selecting and depositing a third chromogen on the specimen; and
rinsing the specimen, such that a portion of a first cell type is stained a different color than a portion of a second cell type, and both the first cell type and the second cell type are stained different colors than a portion of a third cell type.

11. The process of claim 10, wherein the step of selecting and depositing the third one of the plurality of selectively binding agents includes selecting a cytokeratin 20 antibody.

12. The process of claims 10 and 11, wherein the step of deposition the cocktail includes preparing a mixture of a CD44 monoclonal antibody and a p53 monoclonal antibody.

13. The process of claims 10-12, wherein the step of selecting a first chromogen includes selecting a diaminobenzidine chromogen.

14. The process of claims 10-13, wherein the step of selecting a second chromogen includes selecting a blue chromogen.

15. The process of claims 10-14, wherein the step of selecting and deposition the third chromogen includes selecting a red chromogen.

16. The process of claims 10-15, further comprising:
counterstaining the specimen.

17. The process of claim 16, wherein the step of counterstaining deposits hematoxylin on the specimen.

18. The process of claims 10-17, wherein the process steps are automated.

19. The process of claim 18 comprising:
analyzing after the other steps of the process are completed, automatically, using an automated cytological analyzer whether a threshold value of the analyzer indicates the presence or absence of abnormal cells in field of view of the specimen, when observed under magnification by an optical system of the analyzer.
